(19) United States
(12) Patent Application Publication
(10) Pub. No.: US 2005/0194012 A1
(43) Pub. Date: Sep. 8, 2005

(54) Wearable jacket for diagnosis and endoscope system employing wearable jacket

(75) Inventors: Eiichi Ito, Tokyo (JP); Mitsuhiro Matsumoto, Tokyo (JP); Koji Tsuda, Saitama-ken (JP); Masayuki Honjo, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/072,524

(22) Filed: Mar. 7, 2005

(30) Foreign Application Priority Data
Mar. 8, 2004 (JP) 2004-064144

(51) Int. Cl. A61B 1/04; A61B 1/06; A61B 5/04; A61B 5/08; A61B 5/00; A61B 19/00

(52) U.S. Cl. 128/899; 600/160; 600/549; 600/529; 600/509; 128/903

(57) ABSTRACT

A wearable jacket includes a 2D-DST substrate shaped to cover a body of a subject person. The 2D-DST substrate include a conductive sheet, and a plurality of communication modules distributed on the conductive sheet. At least one communication module is provided with a connector portion to which one of a predetermined types of functional unit is electrically connected.

### BACKGROUND OF THE INVENTION

The present invention relates to a wearable jacket for diagnosing a subject person and an endoscope system employing such a wearable jacket.

Conventionally, when a human body of the subject is observed, an electronic endoscope is typically used. The electronic endoscope is provided with cables and optical fibers inside a flexible tube section of a scope, and at a tip thereof, an imaging element such ac a CCD (Charge Coupled Device) is fixed. Such an endoscope is configured such that a relatively long flexible tube is inserted in the human cavity. Therefore, observation using such an endoscope is burden to the subject (examinee). Further, it is difficult to insert such an endoscope in a thin, long and meandering portion such as intestine.

Recently, in order to decrease the burden to the subject, a system employing a capsule type endoscope has been suggested. With use of such a capsule type endoscope, it becomes easy to observe the intestine or the like.

An example of the endoscope system employing the capsule endoscope is described in Japanese Patent Provisional Publication P2003-19111A. According to the endoscope system disclosed in the above publication, a belt having a plurality of antennas is wound around the subject (examinee). The capsule type endoscope outputs a radio wave and the belt is configured to receive the radio wave, which is used for detecting a location of the capsule type endoscope. In this publication, the capsule type endoscope is described to measure condition inside the human cavity or to capture images of inner walls of the human cavity.

According to the above publication, each antenna mounted on the belt is fixed to the belt when the belt is released as a product. In such a configuration, however, the problems as indicated below would arise.

Depending on subject people (i.e., depending on a symptom), a portion to be observed specifically by use of the capsule endoscope is different. If the antennas are fixed to the belt, for a certain subject person, the belt (i.e., the arrangement of the antennas thereon) may be appropriate, however, for another subject person, the belt, or the arrangement of the antennas may be inappropriate. If the antennas are appropriately arranged (i.e., the number of the antennas are relatively small or arrangement does not meet the observation), the radio wave transmitted by the capsule endoscope may not be received by the antennas. In such a case, an image may not be displayed clearly on a display device. Therefore, the belt as described in the publication cannot be used for various subject persons having various symptoms.

### SUMMARY OF THE INVENTION

The present invention is advantageous in that an improved endoscope system employing a capsule endoscope and overcoming the above problems is provided. That is, according to the improved endoscope system, a wearable jacket is provided. The wearable jacket mounts thereon a plurality of antennas, arrangement thereof can be changed in accordance with a subject person. The present invention is further advantageous in that a wearable jacket used for diagnosing various subjects can be provided.

According to an aspect of the invention, there is provided a wearable jacket, which includes a 2D-DST substrate shaped to cover a body of a subject person. The 2D-DST substrate include a conductive sheet, and a plurality of communication modules distributed on the conductive sheet, at least one communication module being provided with a connector portion to which one of predetermined types of a functional unit is electrically connected.

According to another aspect of the invention, there is provided a wearable jacket system, which is provided with a 2D-DST substrate shaped to cover a body of a subject person, the 2D-DST substrate including a conductive sheet and a plurality of communication modules distributed on the conductive sheet, at least one communication module being provided with a connector portion, and a plurality of types of functional units, any one of predetermined types of functional unit being electrically connected to the connector portion of the at least one communication module.

Optionally, the plurality of types of functional units may include one of a body temperature sensor, a sensor for measuring a breathing rate, a cardiac rate or blood pressure, a blood flow sensor, a sensor for measuring oxygen saturation degree, a sensor for detecting sweat, a sensor detecting uric acid level, a sensor for detecting occurrence of bleeding, electrodes for cardiographic measurement, and a communication unit that receives a signal transmitted by an external device.

Further optionally, the wearable jacket system may include a controlling system that determines a transmission path of data in accordance with a 2D-DST technology, the controlling system storing the data received via the transmission path to a predetermined recording medium.

In a particular case, the controlling system may include a data conversion system that converts the received data so as to be displayed on a display device.

Optionally, the controlling system may further include an interface to which a removable recording medium is connected, the controlling system storing the data in the removable recording medium via the interface.

Further optionally, the controlling system may be capable of communicating an external device via a wireless communication, the data being transmitted to the external device via the wireless communication.

Still optionally, the plurality of types of units may have different pieces of ID information, and the at least one of the plurality of communication modules may obtain the ID information of a unit connected to the at least one of the plurality of communication modules and transmit the obtained ID information along with ID information of the at least one of the plurality of communication modules.

Optionally, if at least one of the plurality of communication modules is connected with a communication unit capable of receiving signal transmitted by an external device, the controlling system may control the at least one of the plurality of communication modules to transmit the signal received from the external device and body information other than the signal transmitted by the external device at every predetermined period.

Alternatively, if at least one of the plurality of communication modules is connected with a communication unit capable of receiving signal transmitted by an external device, the controlling system may control the at least one of the plurality of communication modules to transmit the signal received from the external device and body information other than the signal transmitted by the external device periodically at different timings, respectively.

Further, the controlling system may control the at least one of the plurality of communication modules to transmit the signal received from the external device at every first predetermined period and to transmit the body information other than the signal transmitted from the external device at every second predetermined period, which is longer than the first predetermined period.

In a particular case, the controlling system may select an optimum communication module mounting a communication unit from among the plurality of communication modules at every third predetermined period which is longer that the predetermine period.

Still optionally, the plurality of types of functional units may include a communication unit that is capable of at least one of a function of transmitting a signal carrying predetermined information to an external device and another function of receiving a signal transmitted by the external device.

In the above case, the predetermined information may include at least one of a signal that supplies an electrical power to the external device and a signal that controls tan operation of the external device.

According to another aspect of the invention, there is provided an endoscope system which includes a capsule endoscope having a communication function and a wearable jacket system. The capsule endoscope may include an imaging device that captures an image, and a wireless communicating system that transmits image data representing the captured image toward the wearable jacket. The wearable s jacket ystem may include a 2D-DST substrate shaped to cover a body of a subject person, the 2D-DST substrate including a conductive sheet and a plurality of communication modules distributed on the conductive sheet, at least one communication module being provided with a connector portion, a plurality of types of functional units, any one of predetermined types of functional unit is electrically connected to the connector portion of the at least one communication module, the at least one communication module including a reception module which is a communication module connected with a communication unit via the connector portion, the communication unit being capable of receiving the image data transmitted by the wireless communicating system of the capsule endoscope, and a displaying system that displays an image represented by the signal received by the communication module.

### BRIEF DESCRIPTION OF THE ACCOMPANYING DRAWINGS

FIG. 1 schematically shows a configuration of an endoscope system according to an embodiment of the invention;

FIG. 2 schematically shows a configuration of a capsule endoscope employed in the endoscope system according to the embodiment of the invention;

FIG. 3 shows a cross sectional structure of a part of a diagnosis jacket employed in the endoscope system according to the embodiment of the invention;

FIG. 4 is a block diagram showing a configuration of a communication module and an antenna unit coupled to the communication module;

FIG. 5 is a block diagram showing a configuration of a communication module and a sensor unit coupled to the communication module;

FIG. 6 is a block diagram showing a control unit employed in the endoscope system shown in FIG. 1;

FIG. 7 is a flowchart illustrating a data obtaining procedure executed by the control unit shown in FIG. 6 according to the embodiment of the invention;

FIG. 8 is a detailed flowchart illustrating a reception module selecting procedure which is executed in the flowchart shown in FIG. 7;

FIG. 9 shows a cross sectional structure of a jacket according to a modification of the first embodiment of the invention;

FIG. 10 shows a cross sectional structure of a jacket according to another modification of the first embodiment of the invention;

FIG. 11 is a block diagram showing a configuration of a communication module and another antenna unit coupled to the communication module;

FIG. 12 is a flowchart illustrating a data obtaining procedure executed by the control unit shown in FIG. 6 according to a second embodiment of the invention; and

FIG. 13 is a flowchart illustrating a data obtaining procedure executed by the control unit shown in FIG. 6 according to a third embodiment of the invention.

### DETAIL DESCRIPTION OF THE EMBODIMENTS

Referring now to the accompanying drawings, embodiments and modifications of the endoscope system will be described.

General Overview

The endoscope system according to the invention includes a diagnosis jacket. The diagnosis jacket is provided with circuitry to obtain various data of a subject person, or examinee without using lead wires, cables or copper patterns. The obtainable data may include body functions (e.g., pulse, blood pressure, temperature etc.) of the subject and images of body cavities. The diagnosis jacket is configured to be flexible and have durability, light weight, and further realizes freedom of design, higher density of antenna arrangement, and enhances the acquisition of image signals with a high S/N ratio.

### First Embodiment

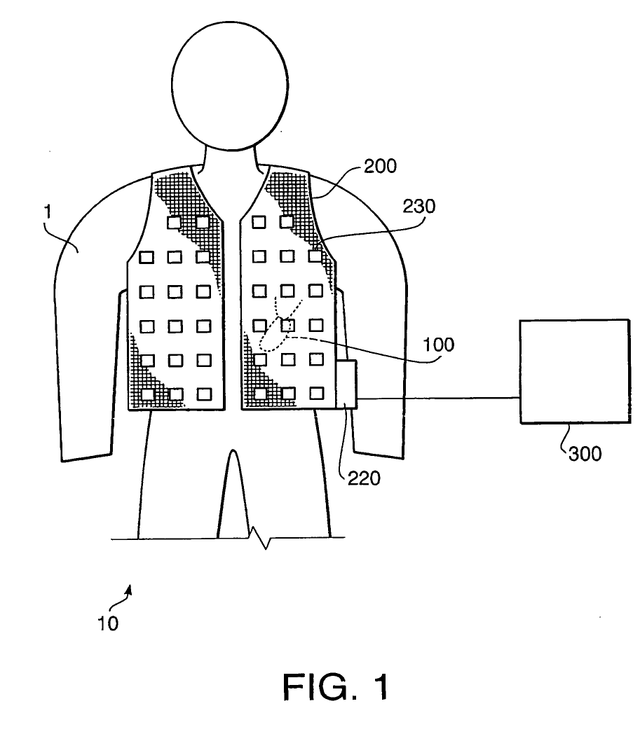

FIG. 1 schematically shows a configuration of the endoscope system 10 according to an embodiment of the invention. The endoscope system 10 is used, for example, to obtain the body functions (e.g., pulse, blood pressure, temperature etc.) and/or image information of body cavities and the like of a subject 1. Such data is used for diagnosing the subject 1.

As shown in FIG. 1, the endoscope system 10 includes a capsule endoscope 100 which is inserted (swallowed) inside the subject 1, the diagnosis jacket 200, and a personal computer (PC) 300. The capsule endoscope 100 captures images inside the subject 1 and outputs image data by radio. The diagnosis jacket 200 is provided with a plurality of antennas and circuits, and receives the image data output by the capsule endoscope 100. The diagnosis jacket 200 transmits the obtained signal and data related to the body functions to the PC 300. The PC 300 is provided with a display, which displays the data (e.g., image data) received from the diagnosis jacket 200. In this specification, a term body function information includes information related to the body function of the subject 1 such as the body temperature, blood pressure, and the like, while a term body information includes at least one of image information captured by the capsule endoscope 100 and the body function information. Acquisition of the body function information will be described in detail later.

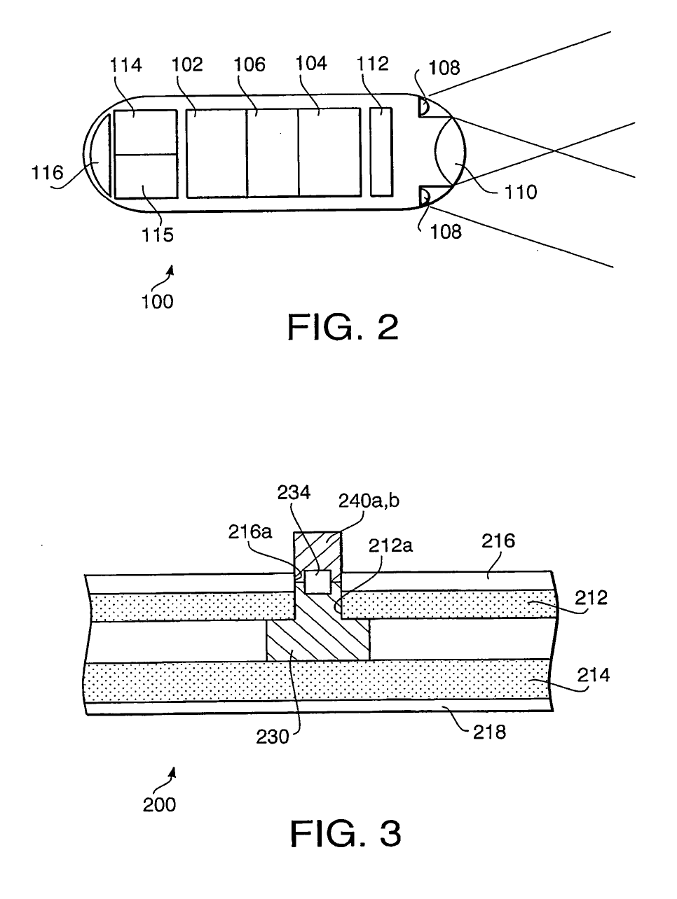

FIG. 2 is a block diagram of the capsule endoscope 100 employed in the endoscope system 10 according to the embodiment. The capsule endoscope 100 has a shape of very small capsule, which can enter thin, long and meandering portions (e.g., a bowel) easily and capture images thereof. The capsule endoscope 100 is provided with a power unit 102 supplying power to each component of the capsule endoscope 100, a control unit 104 that controls entire operation of the capsule endoscope 100, a memory 106 that stores various pieces of data, a pair of illuminating units 108 used for illuminating walls of body cavity, an objective optical system 110 that converges received light to form an image on a solid-state imaging device 112 that captures images of the body cavity, a transmitting unit 114 for transmitting a radio wave carrying image data, a receiving unit 115 for receiving a radio wave transmitted from external devices, and antenna unit 116 from which the radio wave propagates.

When powered on and put into a body cavity of the subject 1, the capsule endoscope 100 illuminates inside the body cavity with the pair of illuminating units 108. The light reflected by walls of the body cavity is incident on the objective optical system 110. The objective optical system 110 and the solid sate imaging device 112 are arranged such that the objective optical system 110 forms an image on the light receiving surface of the solid state imaging device 112. The solid state image receiving device 112 applies a photoelectric conversion to the received optical image to generate an image signal corresponding to the optical image. The control unit 104 controls the transmitting unit 114 to superimpose the thus generated image signal onto a predetermined frequency signal by modulation, and transmit the modulated signal to outside through the antenna unit 116. According to the embodiment, the signal output from the antenna unit 116 is received by the diagnosis jacket 200.

It should be noted that the receiving unit 115 receives the radio wave from an external device, and based on signals represented by the received radio wave, the control unit 104 controls the illuminating units 108 (e.g., ON/OFF control) and other operations of the capsule endoscope 100.

Next, the structure and operation of the diagnosis jacket 200 will be described in detail.

The diagnosis jacket 200 is a wearable jacket which is formed to cover a part of the upper body of the subject 1. It should be noted that the diagnosis jacket 200 may be formed in various shapes and designs. For example, in FIG. 1, a vest type jacket 200 is shown, which is only exemplary design, and a so-called jacket having sleeves can also be used. Since the diagnosis jacket 200 is used for receiving the radio wave transmitted from the capsule endoscope 100 and further used for measuring body functions, it is important that the jacket 200 fits the outer shape of the subject 1.

The diagnosis jacket 200 is provided with a plurality of communication modules 230 distributed therein, which constitute a circuitry for obtaining the image signal transmitted by the capsule endoscope 100, a circuitry for transmitting electromagnetic waves for supplying electrical power and for transmitting control signals, and a circuitry for obtaining body functions of the subject 1. The diagnosis jacket 200 also has a control unit 220 which is located at a waist portion of the subject 1 when wore, and controls the entire operation of the circuitries provided to the diagnosis jacket 200.

FIG. 3 is a partial cross-sectional side view of the diagnosis jacket 200. The diagnosis jacket 200 employs a 2D-DST (two-dimensional diffusive signal transmission) technology, which is laid open in a web site <http://www.utri.co.jp/venture/venture2.html>, in Japanese Patent Provisional Publication No. P2003-18882A. According to the 2D-DST technology, a 2D-DST substrate is configures such that a plurality of chips are distributed between two signal layers so that adjacent chips are locally and electrically connected with each other. Then, data is relayed across the plurality of chips from an origin to a destination by packets. According to the embodiment, the diagnosis jacket 200, which is the 2D-DST substrate in this case, is provided with two conductive sheets 212 and 214, and insulating sheets 216 and 218 for insulating the two conductive sheets 212 and 214 from outside. Between the conductive sheets 212 and 214, a plurality of communication modules 230 are distributed as schematically shown in FIG. 1.

Each of the two layers of conductive sheets 212 and 214 has flexibility and conductivity. Each of the conductive sheets 212 and 214 is formed to be a vest type jacket covering the chest and waist portion and back portion of the subject 1. The conductive sheets 212 and 214 are spaced from each other with a predetermined clearance, having the communication module 230 provided therebetween, not shown insulating layer and/or insulating sheet stacked therebetween. Thus, the conductive sheets 212 and 214 are stacked with electrically insulated from each other. The conductive sheet 212 is on the subject side, while the conductive sheet 214 is on the outer side. In other words, the conductive sheet 212 is a backside sheet of the diagnosis jacket 200, while the conductive sheet 214 is a front side sheet of the diagnosis jacket 200.

The insulating sheet 216 is a flexible sheet having an insulating property. The insulating sheet 216 is shaped and provided to cover the outer surface (i.e., a surface opposite to the surface facing the conductive sheet 214) of the conductive sheet 212. The insulating sheet 216 is made of insulating rubber, insulating film or cloth having an insulating property. Insulating sheet 218 is also a flexible sheet having the insulating property, similar to the insulating sheet 216. The insulating sheet 218 is shaped and provided to cover the outer surface (i.e., a surface opposite to the surface facing the conductive sheet 212) of the conductive sheet 214. Since the insulating sheets 216 and 218 are provided, even if an electrical current flows through the conductive sheet 212 or 214, the outside of the diagnosis jacket 200 is insulated from the conductive sheets 212 and 214, and no electrical currents leak outside.

Next, the communication module 230, and an antenna unit 240a and a sensor unit 240b, each of which can be detachably attached to the communication module, will be described.

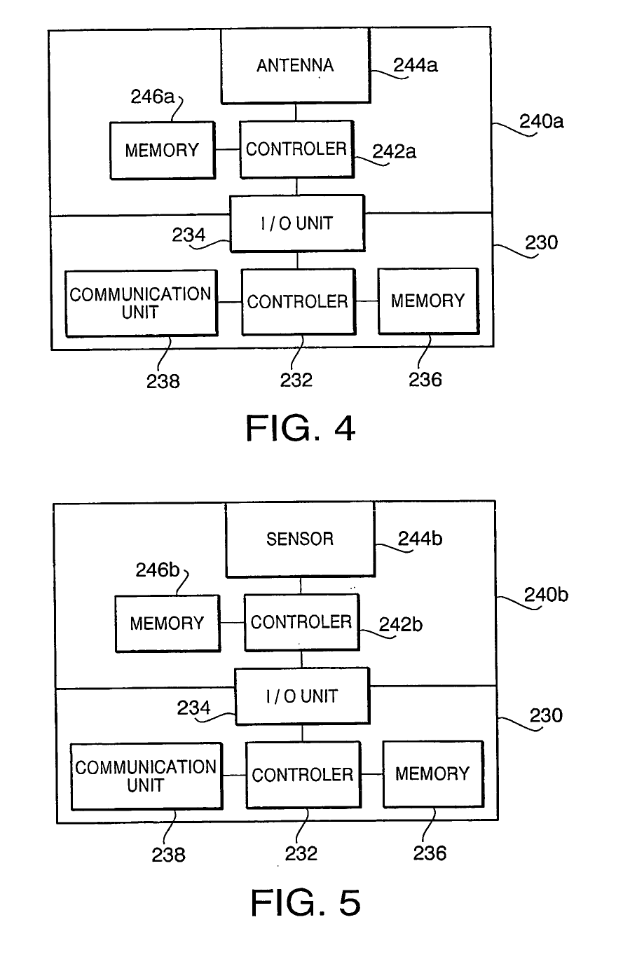

FIG. 4 is a block diagram showing a configuration of the antenna-attached communication module 230 with antenna unit 240a being attached (hereinafter, such a communication module will be occasionally referred to as an antenna-attached communication module 230). FIG. 5 is a block diagram showing configuration of the antenna-attached communication module 230 with sensor unit 240b being attached (hereinafter, such a communication module will be occasionally referred to as a sensor-attached communication module 230).

The communication module 230 includes a controller 232 that controls an entire operation of the communication module 230, an I/O unit 234 that allows a connection (mechanically and electrically) of the communication module 230 with the antenna unit 240a or the sensor unit 240b, a memory 236 for storing various pieces of data including ID information of the communication module 230 and the attached antenna unit 240a or the sensor unit 240b, and a communication unit 238 that executes a communication with adjacent communication modules.

The communication module 230 to which the antenna unit 240a or the sensor unit 240b is not attached functions as a relaying point when a signal (packets) is transmitted in accordance with the 2D-DST technology to a destination. That is, the communication module 230 itself does not have a function to obtain data from the subject 1, but only to relay signals.

According to the first embodiment, the communication module 230, which basically functions as a relaying device, is provided with the I/O unit 234 that allows the communication module 230 to be coupled to various functional units. When an appropriate unit is coupled to the communication module 230 through the I/O unit 234, a corresponding function is implemented. Thus, the operator can customize the diagnosis jacket 200 (i.e., the circuitry in the diagnosis jacket 200) by attaching various units to respective communication modules 230 of the diagnosis jacket 200. Therefore, the operator can construct the circuitries of the diagnosis jacket 200 so that a necessary diagnosis can be performed for each subject 1. It should be noted that, by attaching a functional unit to a communication module 230, the function is added to the communication module 230, and it also functions as the relaying device.

Next, the antenna unit 240a will be described in detail. As shown in FIG. 4, the antenna unit 240a includes a controller 242a that control the entire operation of the antenna unit 242a, an antenna 244a that transmits/receives a radio wave having a predetermined frequency, and a memory 246a that stores various pieces of data including the ID information of itself.

The antenna unit 240a mainly functions to receive an image signal transmitted by the capsule endoscope 100 and to transmit a power supply signal that generates an electrical power inside the capsule endoscope 100 and a control signal carrying control information reflecting the operator's intention to the capsule endoscope 100, through the antenna 244a. Since the antenna unit 240a can transmit the power supply signal, the capsule endoscope 100 can operate for a relatively long period although it mounts only a small battery. Further, by transmitting the control signal, the operator can remotely control the operation of the capsule endoscope 100.

According to the embodiment, the antenna unit 240a has a function of an A/D converter for converting the received signal (analog signal) to a digital signal, and a function of a D/A converter for converting a digital signal to be transmitted into an analog signal to be applied to the antenna 244a. Although the antenna unit 240a according to the embodiment has both the signal receiving and transmitting functions, the invention need not be limited to such a configuration, and only one of receiving and transmitting functions may be used.

It should be noted that, since the capsule endoscope 100 is mainly used for capturing images inside the bowel, it is preferable that a plurality of antenna units 240a are concentrically arranged at a stomach area of the subject 1 (i.e., a plurality of antenna units 240a are attached to the communication modules 230 located at the stomach area of the subject 1).

As shown in FIG. 3, at a position of each communication module 230, the conductive sheet 212 is formed with an opening 212a. Further, on the insulating sheet 216, an opening 216a is formed at a position corresponding to each of the opening 212a. Therefore, through the openings 216a and 212a, the I/O unit 234 is exposed to outside the diagnosis jacket 200. As a result of the structure, the antenna unit 240a can easily be attached to the communication module 230. Since the antenna 244a as attached to the communication unit 230 is exposed to the subject 1, good transmission/reception of the radio wave between the antenna 244a and the capsule endoscope 100 is achieved.

It should be noted that the antenna 244a should be exposed to the subject 1 (i.e., the capsule endoscope side). Since the layer 212 is a conductive layer, if the antenna 244a is not exposed, the radio wave is shielded by the conductive layer 212 and cannot be transmitted to or received from the capsule endoscope 100.

Further, according to the diagnosis jacket 200 configured as above, each antenna 244a is exposed to the subject 1, while the conductive sheet 214 is provided on the opposite side. Therefore, the conductive sheet 214 serves as a shield for the radio wave which is transmitted from a direction opposite to the direction in which the radio wave is transmitted from the capsule endoscope 100 (i.e., from an external device located around the subject 1). Thus, unexpected noise directed to the antenna 244a can be shielded by the conductive sheet 214. According to another aspect, the radio wave transmitted from the antenna 244a only propagates toward the capsule endoscope 100, and does not affect devices around the subject 1. Therefore, the antenna 244a can receive the radio wave (image signal) transmitted from the capsule endoscope 100 at a relatively high S/N ratio. Further, the conductive sheet 214 also shields the radio wave transmitted by the capsule endoscope 100 from propagating toward external devices around the subject 1.

In the above-described embodiment, the antenna unit 240a is capable of receiving and transmitting the radio wave. However, the invention need not be limited to such a configuration, and the antenna unit 240a may be modified to have only one of receiving and transmitting functions.

In the embodiment described above, the capsule endoscope 100 and the antenna unit 240a communicate with each other using a radio wave having a predetermined frequency. It is possible to modify this configuration such that the communication is performed using another spatially propagating signal. For example, light waves may be used for communication by employing a photo diode, LED (light emitting diode) or LD (laser diode). In such a case, the antenna 244a may be replaced with the photo diode for receiving the light wave. For the transmission function, the antenna 244a may be replaced with the LED or LD. Of course, if both the transmission/reception functions are implemented, both the photo diode and the LED or LD are to be employed instead of the antenna 244a.

Alternatively, an audio wave may be used as another form of the spatially propagating wave. When the audio wave is utilized, the antenna 244a may be replaced with a supersonic wave receiver. For transmitting the audio wave, the antenna 244a may be replaced with a supersonic transmitter. For reception/transmission, both the supersonic receiver/transmitter may replace the antenna 244a.

Next, the sensor unit 240b will be described in detail with reference to FIG. 5. As shown in FIG. 5, the sensor unit 240b includes a controller 242b that controls the entire operation of the sensor unit 242b, a sensor 244b that obtains body function information, and a memory 246b that stores various pieces of data including the ID information of itself.

The sensor unit 240b mainly serves to obtain body function information, such as the body temperature, breathing rate, cardiac rate and the like using a corresponding sensor. With the function of the sensor unit 240b, the operator can check the body function of the subject 1 while observing the image of the body cavity by use of the capsule endoscope 100. Therefore, if the body condition of the subject 1 turns bad during the observation, it is immediately recognized by the operator, and thus can be treated quickly.

As the sensor 244b, various types of sensors can be used. For example, a temperature sensor for measuring the body temperature, a pressure sensor for measuring the breathing rate, cardiac rate or blood pressure, a Ph sensor for measuring a hydrogen-ion concentration, a uric acid sensor for measuring a uric acid value of sweat, a light sensor for measuring existence/unexistence of bleeding, a supersonic sensor for measuring a blood flow volume, a photo sensor for measuring an oxygen saturation degree, electrodes for cardiology measurement, and the like. The plurality of sensor units 240b have the above sensors, respectively, and arranged at appropriate positions of the diagnosis jacket 200. Further, since the sensor unit 240b can be repositioned easily (simply by detaching from a communication module 230 and attaching to another communication module 230), the senor units 240b can be located at optimum positions for the subject 1.

For example, when the sensor units 240b having the pressure sensor for measuring the cardiac rate are used, they are attached to the communication modules 230 provided at positions of the diagnosis jacket 200 facing a left thorax (close to the heart) of the subject 1.

The sensor unit 240b mounting the temperature sensor is configured such that the sensor 244b in FIG. 5 serves as the temperature sensor such as one using a thermistor. Such a temperature sensor is mainly used for measuring the body temperature (strictly speaking, the body surface temperature) of the subject 1.

The sensor unit 240b mounting the pressure sensor is configured such that the sensor 244b in FIG. 5 serves as the pressure sensor (e.g., a diaphragm type or semiconductor type pressure sensor). When the breathing rate is measured, the pressure of the body surface of the subject 1 is measured with a measuring frequency of 10 through 20 times/minute, and the number of breathing is calculated. When the cardiac rate is measured, the pressure of the body surface is measured with a measuring frequency of 50 through 100 times/minute, and the number of heart beat is obtained. It should be noted that the diagnosis jacket 200 is elasticated so that the sensors mounted thereon are press-contacted against the body surface of the subject 1. With this elasticated configuration, it is possible to press the pressure sensor against a blood vessel running close to the surface of the body to measure the blood pressure.

The sensor unit 240b mounting the supersonic wave sensor is configured such that the sensor 244b in FIG. 5 serves as the supersonic wave sensor, which includes a supersonic wave receiver and transmitter (in this example, the supersonic sensor includes an integrally combined supersonic receiver/transmitter). The supersonic sensor emits a supersonic wave into the body cavity of the subject 1, and detects a Doppler shift (i.e., a change of frequency in accordance with the Doppler effect) to calculate the blood flow.

The sensor unit 240b mounting the photo sensor is configured such that the sensor 244b in FIG. 5 serves as the photo sensor, which includes a light source (e.g., LED or LD) and the photo diode. In this embodiment, the sensor provided with both the light source and photo diode will be referred to as the photo sensor.

The photo sensor is used for measuring the degree of oxygen saturation in the blood, making use of the characteristic of the blood such that an absorption factor for infrared light of hemoglobin varies as the oxygen saturation degree of the hemoglobin in the blood changes. Specifically, the photo sensor functions as a reflection type photo interrupter. For example, from the LED, light is emitted to the blood inside the body cavity. Then, the reflected light is received by the photo diode to determined the status of the reflected light. Based on the detection result, the oxygen saturation degree is calculated.

According to the structure of the sensor unit 240b and the communication module 230, since the sensor 244b is slightly protruded toward the subject 1 from the surface of the diagnosis jacket 200, excellent adhesiveness of the sensor 244b with respect to the subject 1 is obtained. In particular, when the pressure sensor is used as the sensor 244b, it is ensured that the pressure sensor contacts the surface of the subject 1, and accurate measurement can be performed.

When the antenna unit 240a (or the sensor unit 240b) is connected to the communication module 230, the controller 232 of the communication module 230 accesses the memory 246 of the antenna unit 240a (or the memory 246b of the sensor unit 240b) via the I/O unit 234 to retrieve the data including the ID information of the unit. With this configuration, the controller 232 of the communication module 230 recognizes the type of the unit attached thereto. Further, the retrieved data can be transmitted together with the ID information of the communication module 230 to the control unit 220 in accordance with the 2D-DST technology. Therefore, the control unit 220 can recognize the functions added to the communication modules 230, respectively.

Next, the configuration of the control unit 220 that controls the entire operation of the diagnosis jacket 200 will be described. The control unit 220 mainly has a function of controlling the entire operation of the diagnosis jacket 200, and a function as an interface.

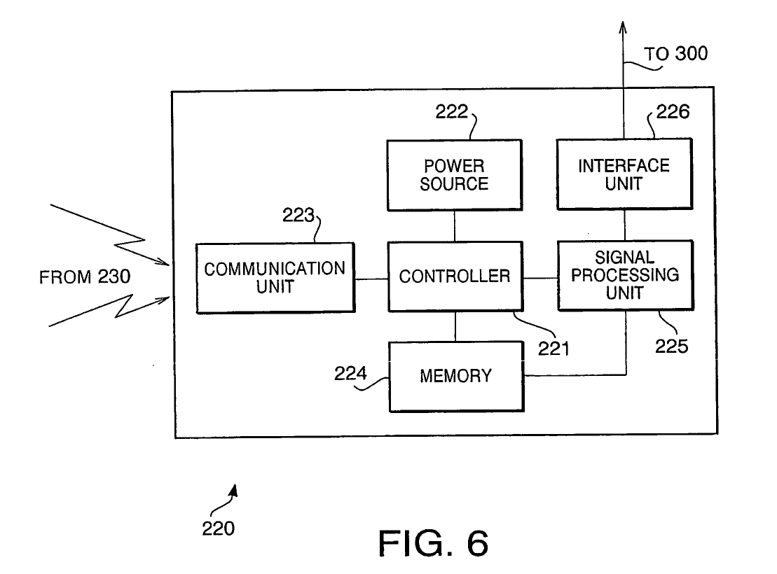

FIG. 6 is a block diagram showing a configuration of the control unit 220. The control unit 220 has a controller 221 which functions as a controller for the entire operation of diagnosis jacket 221, a power source 222 that supplies electrical power to the diagnosis jacket 200, a communication unit 223 that communicates, through the conductive sheet 212 or 214, with the communication modules 230 located close to the control unit 220, a memory 224 for storing various data including control programs and data including the obtained image signal and body information, a signal processing unit 225 that processes the obtained image signal to display an image on the display of the PC 300, and an interface unit 226 through which the control unit 220 is connected with external device and outputs data (e.g., image data and body function data) to the external device. The data obtained by respective communication modules 230 are collected by the control unit 220, which transmits the collected data to the PC 300 so that the operator can view the same on the display of the PC 300.

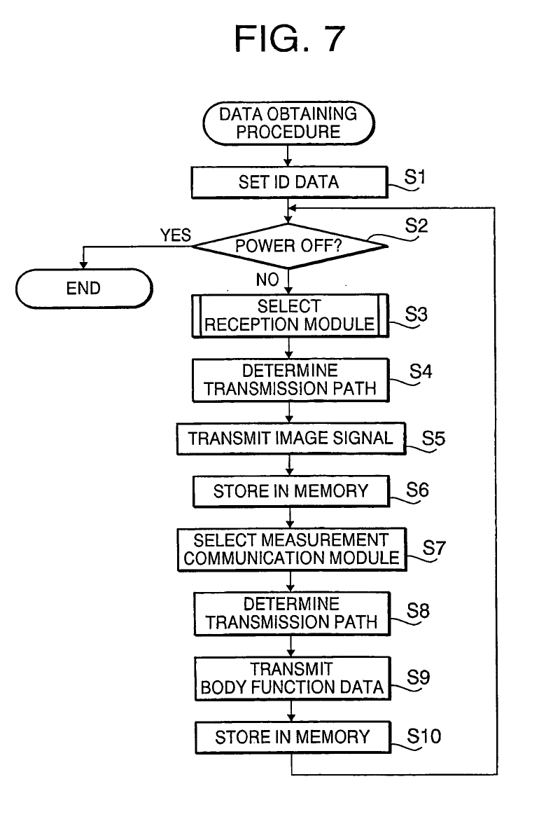

FIG. 7 is a flowchart illustrating a data obtaining procedure which is executed by the control unit 220 (i.e., the controller 221) to obtain various pieces of data including the image data and body function data. In the following description, as an example, it is assumed that a plurality of antenna units 240a and a plurality of sensor units 240b are mounted to the diagnosis jacket 200.

When a power switch (not shown) of the control unit 220 is turned ON, the power source 222 supplies the electrical power to the control unit 220, thereby the control unit 220 starts its operation. Then, the controller 221 can communicate with the communication modules 230 in accordance with the 2D-DST technology. Each communication module 230 operates in accordance with the algorithm (i.e., program) stored in the controller 232 to obtain the ID information, and transmits the ID information to the control unit 220 (S1). The controller 221 can distinguish respective communication modules based on the ID information.

When the ID information setting process (S1) is finished in each communication module 230, the controller 221 judges whether the power source is switched ON or OFF (S2). If the power switch is switched OFF (S2: YES), the controller 221 finishes the procedure shown in FIG. 7. When the power switch is ON (S2: NO), controller 221 advances the procedure to S3.

In S3, the controller 221 selects an antenna-attached communication module 230 that receives the radio wave output by the capsule endoscope 100. In the following description, the antenna-attached communication module 230 that receives the radio wave is occasionally referred to as a reception module.

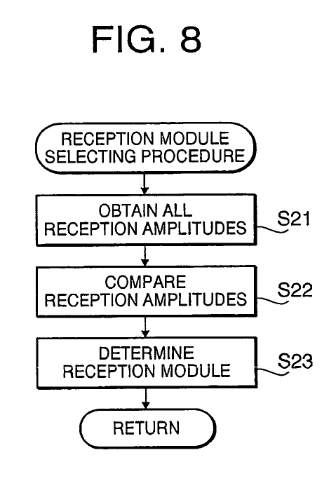

FIG. 8 shows a flowchart illustrating the reception module selecting procedure which is a subroutine called in S3 of the flowchart shown in FIG. 7.

When the reception module selecting operation is called, the controller 221 obtains reception amplitude data representing a signal reception amplitude by each antenna-attached communication module 230 (i.e., by the antenna 244a) regarding the image signal transmitted by the capsule endoscope 100 (S21). In S22, the signal reception amplitude data of all the antenna-attached communication modules 230 distributed over the diagnosis jacket 200 are compared to determined the antenna-attached communication module 230 having the greatest amplitude. In S23, the controller 221 selects the antenna-attached communication module 230 that has determined to have the greatest signal reception amplitude in S21 is selected as the module to receive the image signal transmitted from the capsule endoscope 100. Then, the controller 221 controls the selected antenna-attached communication module 230 to received the radio wave transmitted from the capsule endoscope 100. When the antenna-attached communication module 230 to receive the signal is determined in S23, the reception module selecting procedure is finished, and process proceeds to S4 of FIG. 7. It should be noted that the antenna-attached communication module 230 that receives the radio wave from the capsule endoscope 100 demodulates the received signal to obtain the image signal carried by the radio wave.

The antenna-attached communication module 230 which is currently set to serve as the reception module transmits, under control of the controller 221, a radio wave for supplying electrical power to the capsule endoscope 100 at a predetermined timing. Since the capsule endoscope 100 is supplied with the electrical power, it can operate for a relatively long period. It should be noted that, although the reception module is used to transmit the radio wave for supplying the electrical power to the capsule endoscope in the embodiment, it is possible that another communication module is used for this purpose that is not currently used for receiving the radio wave from the capsule endoscope 100. Alternatively, the antenna-attached communication modules 230 may include modules only for supplying the electrical power to the capsule endoscope 100.

In S4, the controller 221 determines a minimum signal transmission path which is one of paths defined by connecting the communication modules 230 from the selected reception module to the controller 221, and having the shortest path length. When the transmission path is determined, the image signal demodulated and obtained by the reception module is transmitted along the determined path and reaches the control unit 220 (S5). The controller 221 stores the thus received image signal in the memory 224 (S6). The image signal stored in the memory 224 is, under control of the controller 221, processed by the signal processing unit 225 and converted into a video signal, which is transmitted to the PC 300 via the interface 226. Thus, on the display of the PC 300, the image of the body cavity of the subject 1 is displayed.

The controller 221 selects sensor-attached communication modules 230 to obtain body functions of the subject 1 (S7). The sensor-attached communication modules 230 are selected in accordance with a predetermined order. For example, when step S7 is executed first time, a sensor-attached communication module 230 having the temperature sensor is selected, and thereafter, at each execution of step S7, sensor-attached communication modules 230 having the pressure sensor, supersonic wave sensor, photo sensor, and electrodes are selected respectively.

Alternatively, the surface of the diagnosis jacket 200 is divided into a plurality of areas (e.g., chest area, stomach area, etc.), and the sensor-attached communication modules 230 in different area may be selected at every execution of step S7.

Further alternatively, if the total number of the sensor-attached communication modules 230b provided on the diagnosis jacket 200 is relatively small, all the sensor-attached communication module 230 may be selected at a time.

The controller 232 of the selected sensor-attached communication module 230 calculates a measurement value based on the value detected by the sensor 244b, and stores the measurement value in the memory 236 as body function data of the subject 1.

In S8, the controller 221 determines a minimum transmission path that connects the communication modules 230 from the selected sensor-attached communication module 230 to the controller 221. When the minimum transmission path is determined, the body function data is retrieved form the memory 236, and transmitted along the determined transmission path through the communication modules 230, and reaches the control unit 220 (S9). The body function data as received is stored in the memory 224 (S10). After storing the received body function data, the controller 221 returns to step S2, and thereafter, repeats the above-described steps S2-S10.

According to the embodiment, the body function data stored in the memory 224 is converted into character signal by the processing unit 225, and superimposed on the video signal which is also processed by the processing unit 225. Then, the video signal is transmitted to the PC 300 via the interface unit 226. Thus, on the display of the PC 300, characters indicating the body functions of the subject 1 as well as the image of the body cavity.

When the operator operates an operable member (not shown) for controlling the capsule endoscope 100, the antenna-attached communication module 230 currently selected reception module transmits, under control of the controller 221, a control signal corresponding to the operation of the operable member to the capsule endoscope 100. With this configuration, the operator can control the operation of the capsule endoscope 100. It should be noted that the module that transmits the control signal to the capsule endoscope 100 need not be limited to the currently selected reception module 230, but another antenna-attached communication module 230 which is not currently receiving image signal can be used. Alternatively, a dedicated communication module only for transmitting the control signal may be employed.

It should be noted that the invention need not be limited to the configurations of the above-described embodiment and its modifications, but can be modified further in various ways without departing from the scope of the invention.

For example, in the embodiment, the control unit 220 and the PC 300 are connected with a cable (see FIG. 1). This can be modified such that a wireless connection may be utilized instead of the cable.

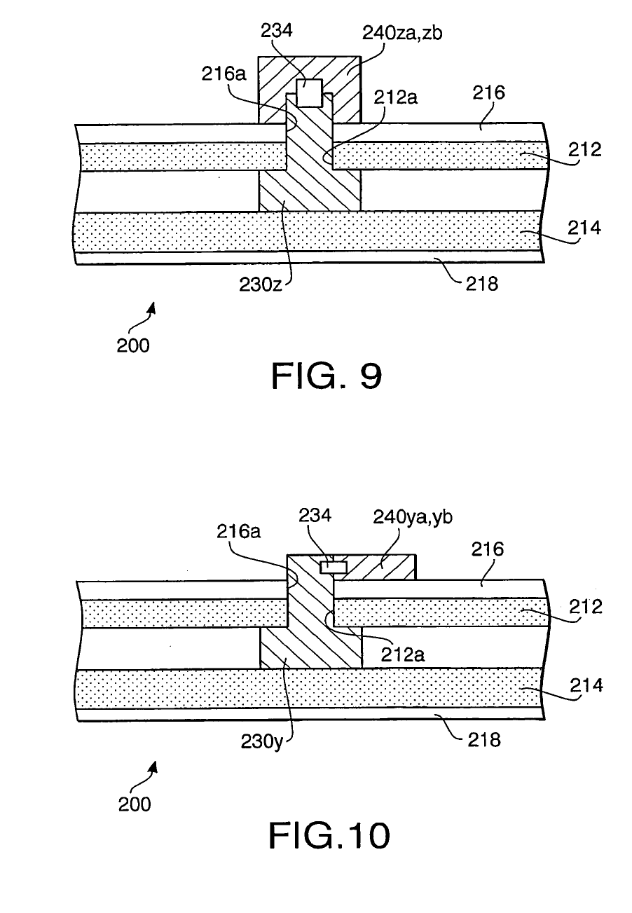

FIG. 9 shows another structure of the diagnosis jacket 200 according to a modification of the above-described embodiment. FIG. 10 shows another modification of the diagnosis jacket 200. In these modifications, structures for connecting the antenna/sensor unit and the communication module 230 are different from that of the above-described embodiment. In the description on the modifications, the elements/structures same as those in the above-described embodiment are referred to using the same reference numbers, which will not be described in detail for the brevity.

In FIG. 9, a communication module 230z is indicated. The communication module 230z is configured such that a part thereof protrudes toward the subject 1 with respect to the outer surface of the insulating sheet 216, and the I/O unit 234 is provided at the tip of the protruded part of the communication module 230z. Further, an antenna unit 240za or a sensor unit 240zb coupled to the communication module 230z is formed to fully cover the protruded part of the communication module 230z. When the antenna unit 240za (or the sensor unit 240zb) is coupled to the communication module 230z, as show in FIG. 9, the protruded part is covered by the coupled unit 240za (or 240zb) in all the directions. Therefore, the antenna unit 240za (or the sensor unit 240zb) is fixed in all the directions except the coupling direction (i.e., up-and-down direction in FIG. 9). With this structure, even if a force is applied to the antenna unit 240za (or the sensor unit 240zb) in the direction other than the up-and-down direction, the antenna unit 240za (or the sensor unit 240zb) will not be detached. That is, with this structure, the mechanical connection between the communication module 230z with the antenna unit 240za (or the sensor unit 240zb) is strengthened. Further, by employing such a structure, it is possible to use a wider antenna, which enhances the signal reception degree.

FIG. 10 shows a communication module 230y. The communication module 230y is configured such that a part thereof protrudes toward the subject 1 with respect to the outer surface of the insulating sheet 216, and the I/O unit 234 is provided at the tip of the protruded part of the communication module 230y. According to this modification, the I/O unit 234 is protruded sideward (i.e., protruded from the protruded part of the communication module 230y in a direction parallel to the surface of the insulating sheet 216). Therefore, according to this structure, an antenna unit 240ya or a sensor unit 240yb is coupled to the I/O unit 234 from the sideward direction. With this structure, it is possible to suppress the thickness (i.e., the length in up-and-down direction in FIG. 10) of the diagnosis jacket 200 when the antenna units 240ya and/or sensor units 240yb are attached.

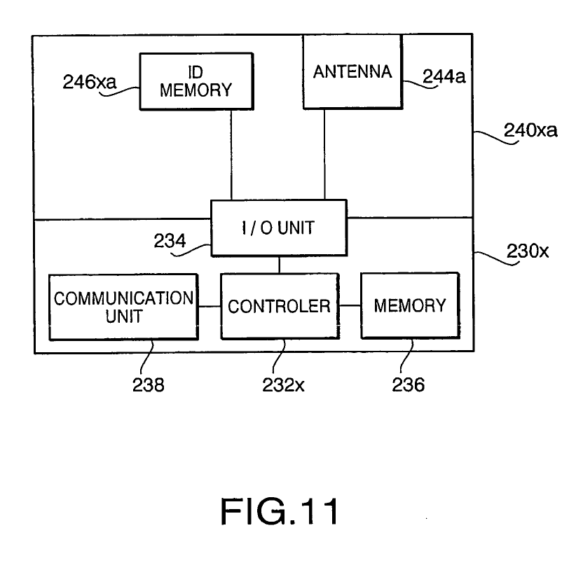

FIG. 11 shows an antenna unit 240xa which is a further modification of the antenna unit 240a shown in FIG. 4. According to this modification, the antenna unit 240xa has an antenna 244a and an ID memory 246xa storing the ID information thereof. As is appreciated, the antenna unit 240xa does not include a controller such as the controller 242a shown in FIG. 4. Since the controller is not included, the antenna unit 240xa can be manufactured at a relatively low cost. When this type of antenna unit 240xa is used, the procedure executed by the controller 242a in the above-described embodiment is executed by a controller 232x of a communication module 230x. For example, the radio wave received by the antenna 240a is transmitted to the communication module 230x via the I/O unit 234. Then, the controller 232x converts the received signal into a digital signal. Further, a signal for supplying the electrical power to the capsule endoscope 100 is converted from the analogue to digital by the controller 232x, and is transmitted to the antenna 244a via the I/O unit 234.

It is of course possible to modify the configuration shown in FIG. 11 such that the antenna 244a may have only the transmitting or receiving function. Further, as the spatially propagating signal, light wave or sonic wave may be used instead of the radio wave.

The configuration shown in FIG. 11 is also applicable to a sensor unit. That is, instead of the antenna 244a, sensor 244b may be used in the structure shown in FIG. 11.

Optionally, the control unit 220 may be provided with a memory card slot so that image data and/or body function data can be stored in a memory card inserted in the card slot.

In the embodiment, the communication modules are arranged on the jacket shaped wearable. The shape of the wearable jacket need not be limited to the vest shape, but the wearable jacket may have various shapes, such as a shape of a belt.

### Second Embodiment

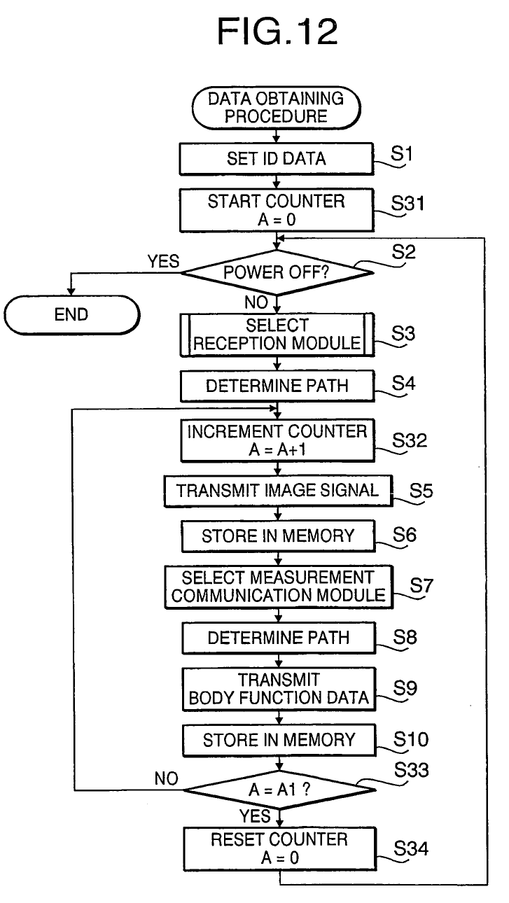

FIG. 12 shows a flowchart illustrating a data obtaining procedure executed by the control unit 220 according to a second embodiment. According to the procedure shown in FIG. 7, the reception module is selected every time the obtained image and the body function data is stored in the memory 224. According to the second embodiment, the reception module is selected at very predetermined timings. In the following description on FIG. 11, the steps which are the same as those in FIG. 7 will be assigned with the same step numbers and description thereof will be omitted for the brevity.

When the power switch of the control unit 220 is ON and the ID data setting process is executed in S1, controller 221 starts a counter A (which has an initial value of zero) in S31. The counter A is referred to in S33, which will be described later. After controller 221 judges whether the power source is OFF in S2, and steps S3 and S4 are executed, the controller 221 increments the counter A by one.

After steps S5 through S10 are executed (i.e., the image signal and the body function data are transmitted to the control unit 220 and stored in the memory 224), the controller 221 judges whether the value of the counter A is equal to a predetermined value A1, which corresponds to a predetermined timing. If the value of the counter A is not equal to A1 (S33: NO), the controller 221 returns the process to S32, increments the counter A by one, and repeats the steps S5 through S10 again.

If the value of the counter A is equal to A1 (S33: YES), the controller 221 resets the counter A (i.e., sets the counter to zero) in S34, and the controller returns to S2.

According to the second embodiment, as described above, a period for selecting the reception module is longer than a period for transmitting the image signal and body function data to the control unit 220.

According to the procedure shown in FIG. 11, the reception module is not selected every time when the image data and body function data are stored in the memory 224, but the selection is made at every predetermined period, which is longer than the period for storing the image data and body function data in the memory 224. Therefore, according to the second embodiment, the reception module selection procedure is executed less frequently and the burden to the controller 221 is decreased in comparison with the first embodiment.

The configuration of the second embodiment is particularly effective when the capsule endoscope 100 moves at a relatively low speed, since it is not necessary to change the reception module so frequently.

### Third Embodiment

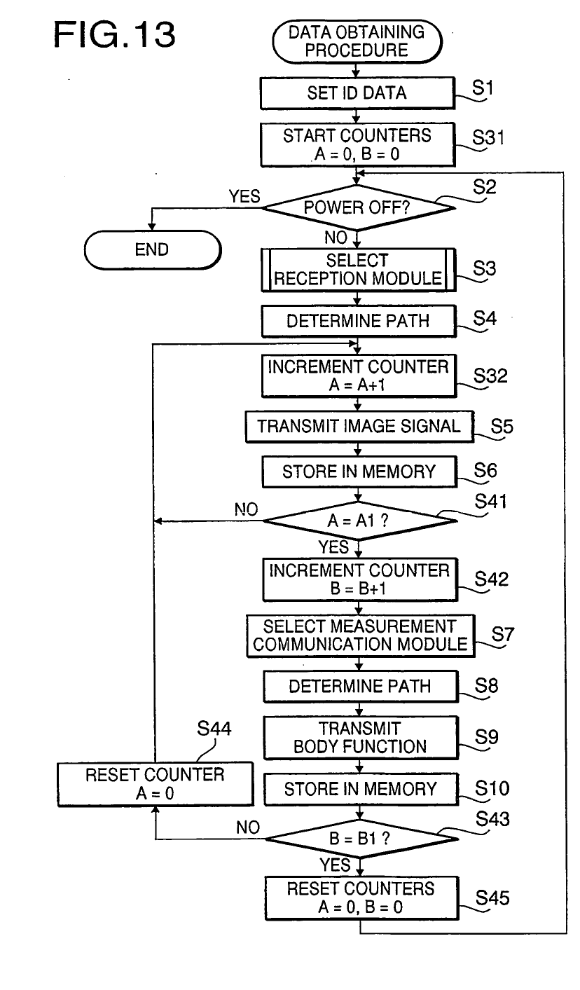

FIG. 13 is a flowchart of the data obtaining procedure according to a third embodiment. According to the procedure shown in FIG. 13, acquisition of the image data, acquisition of the body function data and selection of the reception module are executed at different timings. It should be noted that the steps that are the same as those in FIG. 7 or FIG. 12 will be assigned with the same step numbers and description thereof will be omitted for the brevity.

When the power switch (not shown) is turned ON and the 1D data setting process is executed in S1, controller 221 starts the counters A (initial value=0) and B (initial value=0) in S31. After judgment in S2, reception module selection procedure in S3 and path determining procedure in S4 are finished, the controller 221 increments the counter A by one (S32).

When steps S5 and S6 are finished (i.e., the image signal is transmitted to the control unit 220 and stored in the memory 224), the controller 221 judges whether the value of the counter A is equal to A1 in S41. If the value of the counter A is not A1 (S41: NO), the controller 221 returns to S32, where the counter A is incremented by one (S32), and steps S5 and S6 are executed again to receive the image signal again and stores the received image signal in the memory 224.

If the value of the counter A is equal to A1 (S41: YES), the controller 221 increments the counter B by one (S42), and executes steps S7 through S10 (i.e., the body function data is transmitted to the control unit 220 and stored in the memory 224).

When step S10 is executed, the controller 221 judges whether the value of the counter B is equal to B1 (S43). If the value of the counter B is equal to B1 (S43: YES), the controller 221 resets the values of the counters A and B to the initial values (=0) in S45, and the controller 221 returns the process to S2. In the above example, the interval of transmitting the body function data to the control unit 220 is longer than the interval of transmitting the image signal to the control unit 220, and further the interval of selecting the reception modules is longer than the interval of the selection of the reception module.

According to the third embodiment, the image signal and the body function data are not obtained at the same timing. The interval for obtaining the body function data is longer than the interval for obtaining the image signal. Further, according to the procedure shown in FIG. 12, the interval for selecting the reception module is longer than the timing for storing the body function data in the memory 224.

That is, according to the third embodiment, acquisition of the image data, acquisition of the body function data and selection of the reception module are executed at different timings. In particular, according to the third embodiment, emphasis is laid on the acquisition of the image data, the image data is obtained most frequently. Depending on a situation, it is possible to execute the acquisition of the body function data most frequently.

The present disclosure relates to the subject matter contained in Japanese Patent Application No. 2004064144, filed on Mar. 8, 2004, which is expressly incorporated herein by reference in its entirety.

1. A wearable jacket, comprising:
a 2D-DST substrate shaped to cover a body of a subject person,
the 2D-DST substrate including:
a conductive sheet; and
a plurality of communication modules distributed on the conductive sheet, at least one communication module of the plurality of communication modules being provided with a connector portion to which one of predetermined types of functional unit is electrically connected.

2. A wearable jacket system, comprising:
a 2D-DST substrate shaped to cover a body of a subject person,
the 2D-DST substrate including a conductive sheet and a plurality of communication modules distributed on the conductive sheet, at least one communication module of the plurality of communication modules being provided with a connector portion; and
a plurality of types of functional units, any one of the plurality of predetermined types of functional unit is electrically connected to the connector portion of the at least one communication module.

3. The wearable jacket system according to claim 2, wherein the plurality of types of functional units include one of a body temperature sensor, a sensor for measuring a breathing rate, cardiac rate or blood pressure, a blood flow sensor, a sensor for measuring oxygen saturation degree, a sensor for detecting sweat, a sensor for detecting uric acid level, a sensor for detecting an occurrence of bleeding, electrodes for cardiographic measurement, and a communication unit that receives a signal transmitted by an external device.

4. The wearable jacket system according to claim 2, comprising a controlling system that determines a transmission path of data in accordance with a 2D-DST technology, the controlling system storing data received via the transmission path to a predetermined recording medium.

5. The wearable jacket system according to claim 4, wherein the controlling system includes a data conversion system that converts the received data so as to be displayed on a display device.

6. The wearable jacket system according to claim 4, wherein the controlling system further includes an interface to which a removable recording medium is selectively connected, the controlling system storing the data in the removable recording medium via the interface.

7. The wearable jacket system according to claim 6, wherein the controlling system is capable of communicating an external device via a wireless communication, the data being transmitted to the external device via the wireless communication.

8. The wearable jacket system according to claim 4,
wherein the plurality of types of units have different pieces of ID information,
wherein the at least one communication module of the plurality of communication modules obtains the ID information of a unit connected to the at least one communication module of the plurality of communication modules and transmits the obtained ID information along with ID information of the at least one communication module of the plurality of communication modules.

9. The wearable jacket system according to claim 8,
wherein when at least one communication module of the plurality of communication modules is connected with a communication unit capable of receiving a signal transmitted by an external device, the controlling system controls the at least one communication module of the plurality of communication modules to transmit the signal received from the external device and body information other than the signal transmitted by the external device at every predetermined period.

10. The wearable jacket system according to claim 8,
wherein when at least one communication module of the plurality of communication modules is connected with a communication unit capable of receiving a signal transmitted by an external device, the controlling system controls the at least one communication module of the plurality of communication modules to transmit the signal received from the external device and body information other than the signal transmitted by the external device periodically at different timings, respectively.

11. The wearable jacket system according to claim 10,
wherein the controlling system controls the at least one communication module of the plurality of communication modules to transmit the signal received from the external device at every first predetermined period and to transmit the body information other than the signal transmitted from the external device at every second predetermined period, which is longer than the first predetermined period.

12. The wearable jacket system according to claim 9, wherein the controlling system selects an optimum communication module mounting a communication unit from among the plurality of communication modules at every third predetermined period which is longer that the predetermine period.

13. The wearable jacket system according to claim 2, wherein the plurality of types of functional units include a communication unit that is capable of at least one of a function of transmitting a signal carrying predetermined information to an external device and another function of receiving a signal transmitted by the external device.

14. The wearable jacket system according to claim 13, wherein the predetermined information includes at least one of a signal that supplies electrical power to the external device and a signal that controls an operation of the external device.

15. An endoscope system comprising a capsule endoscope having a communication function and a wearable jacket system,
the capsule endoscope comprising:
an imaging device that captures an image; and
a wireless communicating system that transmits image data representing the captured image toward the wearable jacket system,
the wearable jacket system comprising:
a 2D-DST substrate shaped to cover a body of a subject person, the 2D-DST substrate including a conductive sheet and a plurality of communication modules distributed on the conductive sheet, at least one communication module being provided with a connector portion;
a plurality of types of functional units, any one of the predetermined types of functional units being electrically connected to the connector portion of the at least one communication module, the at least one communication module including a reception module which is a communication module connected with a communication unit via the connector portion, the communication unit being capable of receiving the image data transmitted by the wireless communicating system of the capsule endoscope; and
a displaying system that displays an image represented by the signal received by the communication module.
